(19) United States
(12) Patent Application Publication
(10) Pub. No.: US 2022/0359091 A1
(43) Pub. Date: Nov. 10, 2022

(54) Clinical Pathway Integration and Clinical Decision Support

(71) Applicant: Electronic Caregiver, Inc., Las Cruces, NM (US)

(72) Inventors: Anthony Dohrmann, El Paso, TX (US); Mark Francis, Portland, OR (US); Timothy Washburn, Las Cruces, NM (US)

(21) Appl. No.: 17/735,750

(22) Filed: May 3, 2022

Related U.S. Application Data
(60) Provisional application No. 63/184,060, filed on May 4, 2021.

(51) Int. Cl. G16H 80/00; G06N 3/04

(52) U.S. Cl. CPC G16H 80/00; G06N 3/04

(57) ABSTRACT

Exemplary embodiments provided herein include an intelligent secure networked health messaging system configured by at least one processor to execute instructions stored in memory, the system comprising, a data retention system and a health analytics system, the health analytics system performing asynchronous processing with a patient's computing device and the health analytics system communicatively coupled to a deep neural network, a web services layer providing access to the data retention and the health analytics system, a batching service, wherein an application server layer transmits a request to the web services layer for data, the request processed by the batching service transparently to the patient, the request processed by the batching service transparently to the patient such that the patient can continue to use a patient facing application without disruption, the patient-facing application having an audio sensor and a computer video sensor, the application server layer including a high speed data corridor.

### CROSS-REFERENCE TO RELATED APPLICATIONS

The present application claims the priority benefit of U.S. Provisional Patent Application Ser. No. 63/184,060 filed on May 4, 2021 titled, “Clinical Pathway Integration and Clinical Decision Support,” the disclosure of this application incorporated by reference in its entirety.

### FIELD OF THE TECHNOLOGY

The present technology relates generally to secure health messaging, and more particularly, but not by limitation, to systems and methods for secure health messaging that allow modular subsystem isolation, as well as latency remediation and improved user experiences.

### SUMMARY

Exemplary embodiments provided herein include an intelligent secure networked health messaging system configured by at least one processor to execute instructions stored in memory, the system comprising, a data retention system and a health analytics system, the health analytics system performing asynchronous processing with a patient's computing device and the health analytics system communicatively coupled to a deep neural network, a web services layer providing access to the data retention and the health analytics system, a batching service, wherein an application server layer transmits a request to the web services layer for data, the request processed by the batching service transparently to the patient, the request processed by the batching service transparently to the patient such that the patient can continue to use a patient facing application without disruption, the patient-facing application having an audio sensor and a computer video sensor, the application server layer including a high speed data corridor established between the application server layer and the patient's computing device that provides the patient-facing application that accesses the data retention and the health analytics system and the deep neural network through the web services layer, performs processing based on patient interaction with the patient-facing application, the patient-facing application configured to execute instructions including transmitting an interactive conversational patient interface to the patient's computing device, the deep neural network configured to receive a first input at an input layer, process the first input at one or more hidden layers, generate a first output, transmit the first output to an output layer, and provide the first output to the patient-facing application.

Further exemplary embodiments include providing the first output to the interactive conversational patient interface, the first output generating a first outcome, the first outcome being transmitted to the input layer, processing the first outcome by the one or more hidden layers, generating a second output, transmitting the second output to the output layer, providing the second output to the patient-facing application and the second output generating a second outcome, and the second outcome being transmitted to the input layer. A plurality of outcomes may be processed by the one or more hidden layers for a single patient and a comorbid condition may be determined for the single patient. A plurality of outcomes may be processed for a plurality of patients, in some cases the plurality of patients having a medical condition in common. The number of cycles or iterations through various exemplary embodiments may be virtually infinite and outputs may be provided to or transmitted to places capable of receiving the output other than the patient/user facing application.

According to some exemplary embodiments, the output may include any of a clinically relevant care plan, a reminder, an alert, a survey, a biometric parameter, a biometric parameter out of a predetermined threshold, a response to a survey, medication compliance information, an indicator of daily activity, an indicator of mood, or an indicator of stress. In some exemplary embodiments, threshold alerts are personalized and customizable. The processing by the one or more hidden layers may include using voice, speech, and computer video inputs to analyze signs of changes in health and behavioral status including but not limited to stress, anger, change in speech cadence, slurred speech or coughing. The processing may include determining changes in health and behavioral status including but not limited to anger, substance use, lack of sleep, stress, early onset of dementia or Alzheimer's disease, an adverse reaction to a medication, a stroke, Parkinson's disease, an increased risk of falling, or a lack of balance.

The processing, in various exemplary embodiments, by the one or more hidden layers may include using telemetry information to determine if a patient's behavior has changed in a way which could be indicative of a change in mental, emotional, or physical health and proactively inquiring before a threshold alert is triggered. The patient-facing application with the interactive conversational patient interface may convert response data received by the patient's computing device into an audio file using a cloud-based text-to-speech application capable of being integrated into a web browser based avatar, the avatar being displayed on a display screen within the web browser of the patient's computing device as a three-dimensional electronic image of a human caregiver for a human patient, further comprising the three-dimensional electronic image of the human caregiver providing step-by-step verbal healthcare instructions to the human patient, monitoring a response from the human patient, and providing healthcare advice to the human patient based on the response.

Other exemplary embodiments include the patient-facing application configured to generate a report for a health care provider, receive instructions from the health care provider and deliver the instructions to the human patient by way of the three-dimensional electronic image of a human caregiver. Processing by the one or more hidden layers includes using backpropagation to compute a gradient of a loss function with respect to weights of the neural network for a single input—output. Processing by the one or more hidden layers also includes using each individual node as its own linear regression model, composed of input data, a weight, a bias or threshold, and an output. The one or more hidden layers include generating an insight on a health condition proactively before a statistically significant manifestation of a decline in a health condition.

The first output, in various exemplary embodiments, may cause ordering a home safety inspection for a patient with noted activities of daily living limitations. The first output may also cause ordering a functional strength examination for a patient at risk of falling and/or causing prescribing a walker or a wheelchair.

In some exemplary embodiments, an output may be consumed by the patient and the output may also be consumed by a clinician via a web portal which is how the clinician accesses, interprets and acts upon data. Addison, according to exemplary embodiments, may act as a third user (in addition to user/patient and clinician). Addison's engagement with a patient may vary depending on the output from the data analysis. Additionally, the user/patient may interface with output via Addison on a personal computer (“PC”), tablet, smartphone application, text message (from Addison), and even via mixed (augmented and/or virtual) reality.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1 is a schematic diagram of an exemplary computing architecture that includes a system constructed in accordance with the present disclosure.

FIG. 2 shows an exemplary interactive conversational user interface.

FIG. 3 shows an exemplary deep neural network.

FIG. 4 shows another exemplary deep neural network in the form of a geodesic dome.

Appendices 1 and 2 provide further detail about exemplary embodiments.

### DETAILED DESCRIPTION

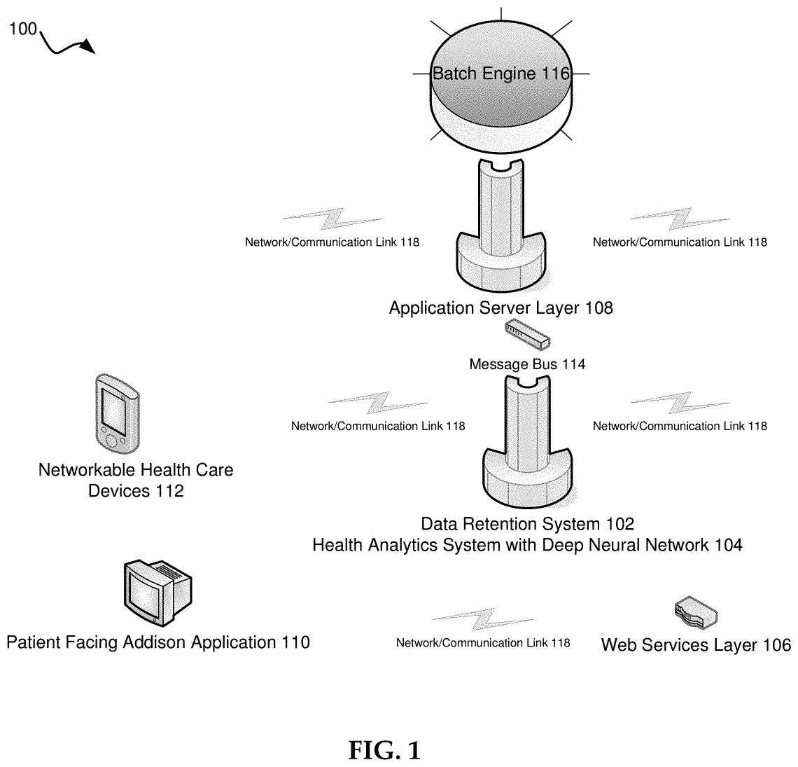

FIG. 1 is a schematic diagram of an example secure health messaging system (hereinafter system 100) for practicing aspects of the present disclosure. The system 100 comprises a data retention system 102, a health analytics system with a deep neural network 104, a web services layer 106, and an application server layer 108 that provides, for example, modeling. Some or all of the activities occur over one or more network/communication links 118, may occur in a cloud computing system and/or in an edge computing system.

In some embodiments, the data retention system 102 and the health analytics system with a deep neural network 104 are in secure isolation from a remainder of the secure messaging system 100 through a security protocol or layer. The data retention system 102 can also provide additional services such as logic, data analysis, risk model analysis, security, data privacy controls, data access controls, disaster recovery for data and web services—just to name a few.

The web services layer 106 generally provides access to the data retention system 102. According to some embodiments, the application server layer 108 is configured to provide a patient or user-facing Addison application 110 that accesses the data retention 102 and the health analytics system with a deep neural network 104 through the web services layer 106.

In one or more embodiments, the application server layer 108 performs asynchronous processing based on user interaction with a health messaging application that processes data from a user via the patient-facing Addison application 110. A health messaging application can reside and execute on the application server layer 108. In other embodiments, the health messaging application may reside with the health analytics system with a deep neural network 104. In another embodiment, the health messaging application can be a client-side, downloadable application. Networkable health care devices 112, according to exemplary embodiments, may include a blood pressure monitor, glucometer, pro health hub, pulse oximeter, various sensors, including third party sensors, motion sensors, fall detection sensors, pressure sensors, telemetry sources, user behavior sources, and/or a thermometer. These devices may transmit information over a network, such as the Internet, to the system 100.

The systems of the present disclosure may implement security features that involve the use of multiple security tokens to provide security in the system 100. Security tokens are used between the web services layer 106 and application server layer 108.

In some embodiments, the system 100 implements an architected message bus 114. Rather than performing the refresh, which could involve data intensive and/or compute or operational intensive procedures by the system 100, the message bus 114 allows the request for refresh to be processed asynchronously by a batching process and provides a means for allowing the patient-facing Addison application to provide a view to the patient, allowing the patient to continue to access data without waiting on the system 100 to complete its refresh.

Also, latency can be remediated at the patient-facing Addison application 110 based on the manner with which the patient-facing Addison application 110 is created and how the data that is displayed through the patient-facing Addison application 110 and how the data is stored and updated. For example, data displayed on the patient-facing Addison application 110 that changes frequently can cause frequent and unwanted refreshing of the entire patient-facing application and interactive graphical patient (or user) interfaces (“GUIs”). The present disclosure provides a solution to this issue by separating what is displayed on the GUI with the actual underlying data. The underlying data displayed on the GUI of the patient-facing Addison application 110 can be updated, as needed, on a segment-by-segment basis (could be defined as a zone of pixels on the display) at a granular level, rather than updating the entire GUI. That is, the GUI that renders the underlying data is programmatically separate from the underlying data cached by the client (e.g., device rendering the GUIs of the patient-facing Addison application 110). Due to this separation, when data being displayed on the GUI changes, re-rendering of the data is performed at a granular level, rather than at the page level. This process represents another example solution that remedies latency and improves user experiences with the patient-facing Addison application 110.

To facilitate these features, the patient facing Addison application 110 will listen on the message bus 114 for an acknowledgement or other confirmation that the background processes to update the user account and/or the patient-facing Addison application have been completed by the application server layer 108. The patient-facing Addison application (or even part thereof) is updated as the system 100 completes its processing. This allows the patient-facing Addison application 110 to be usable, but heavy lifting is being done transparently to the user by the application server layer 108. In sum, these features prevent or reduce latency issues even when an application provided through the patient facing Addison application 110 is “busy.” For example, a re-balance request is executed transparently by the application server layer 108 and batch engine 116. This type of transparent computing behavior by the system 100 allows for asynchronous operation (initiated from the application server layer 108 or message bus 114).

In some embodiments, a batch engine 116 is included in the system 100 and works in the background to process re-balance requests and coordinate a number of services. An example re-balance request would include an instance where a user selectively makes a data request. The batch engine 116 will transparently orchestrate the necessary operations required by the application sever layer 108 in order to obtain data.

According to some embodiments, the batch engine 116 is configured to process requests transparently to a user so that the user can continue to use the user-facing Addison application 110 without disruption. For example, this transparent processing can occur when the application server layer 108 transmits a request to the web services layer 106 for data, and a time required for updating or retrieving the data meets or exceeds a threshold. For example, the threshold might specify that if the request will take more than five seconds to complete, then the batch engine 116 can process the request transparently. The selected threshold can be system configured.

In some embodiments, security of data transmission through the system 100 is improved by use of multiple security tokens. In one embodiment, a security protocol or security token is utilized between the application server layer 108 and the web services layer 106.

For example, feedback responses as described herein may be transmitted back to the data retention system 102 and/or the health analytics system with a deep neural network 104.

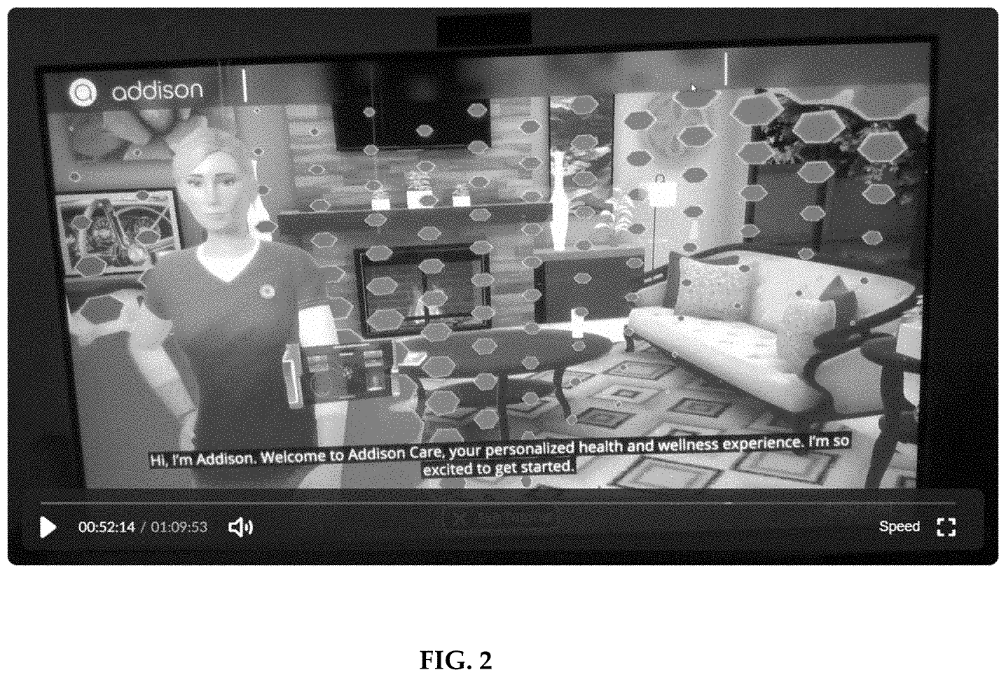

FIG. 2 shows an exemplary interactive conversational user interface.

In some exemplary embodiments, the patient-facing Addison application 110 (FIG. 1) optionally utilizes Unity's eponymous platform. It is used to create two-dimensional, three-dimensional, virtual reality, and augmented reality video games and other simulations. It offers much more flexibility than a browser-based system. In some exemplary embodiments, Unity can be used to make “digital twins”—virtual copies of real-life objects, environments, and people. Unity can be used to create a non-player character or “NPC.” An NPC is any character (such as in a game) that is not controlled by a player. For example, it applies to characters controlled by a gamemaster or referee rather than by another player. Unity also utilizes artificial intelligence tools in its virtual environments. With a digital twin, Unity can collect synthetic data off the simulation to advance its in-real-life (“IRL”) twin. Additionally, in various exemplary embodiments, systems may be plugged into Unity, including facial recognition data, location data, CAD data, computer vision data, natural language processing data, blood pressure monitor data, glucometer data, pro health hub data, pulse oximeter data and/or thermometer data. As described and illustrated herein, in many exemplary embodiments, Addison may represent a NPC of an IRL caregiver (or vice versa). Further, Addison may be controlled in part by a patient or other human.

In some exemplary embodiments, the system 100 includes an Emergency Medical System and/or an Emergency Medical Technician module so emergency personnel can immediately access health care information either on site and/or over a network. The system 100 may also be configured to receive and store a Do Not Resuscitate (“DNR”) order and/or a Last Will and Testament, etc.

The system 100 may also be configured with Addison having the ability to track inventories, such as groceries or medicines, and place automatic reorders. The system 100 may be configured with Addison having the ability to order food through various applications or goods or services through vendors such as Amazon®.

In various exemplary embodiments, the system 100 may be configured with facial recognition capabilities for Addison to determine and interpret a patient's face and changes, including mood and/or possible signs of a stroke or cardiovascular event.

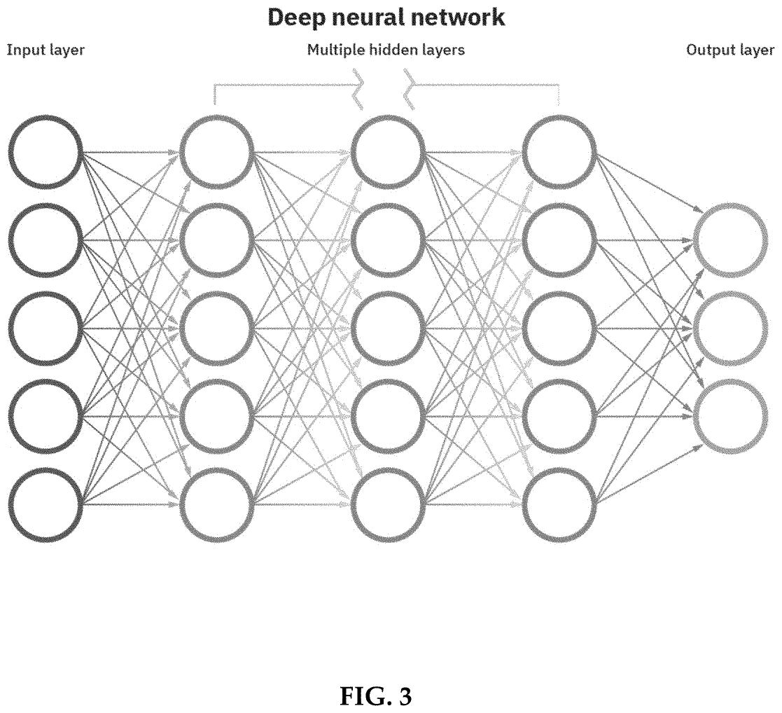

FIG. 3 shows an exemplary deep neural network.

Neural networks, also known as artificial neural networks (ANNs) or simulated neural networks (SNNs), are a subset of machine learning and are at the heart of deep learning algorithms. Their name and structure are inspired by the human brain, mimicking the way that biological neurons signal to one another. Artificial neural networks (ANNs) are comprised of a node layers, containing an input layer, one or more hidden layers, and an output layer. Each node, or artificial neuron, connects to another and has an associated weight and threshold. If the output of any individual node is above the specified threshold value, that node is activated, sending data to the next layer of the network. Otherwise, no data is passed along to the next layer of the network.

Neural networks rely on training data to learn and improve their accuracy over time. However, once these learning algorithms are fine-tuned for accuracy, they are powerful tools in computer science and artificial intelligence, allowing one to classify and cluster data at a high velocity. Tasks in speech recognition or image recognition can take minutes versus hours when compared to the manual identification by human experts. One of the most well-known neural networks is Google's search algorithm.

In some exemplary embodiments, one should view each individual node as its own linear regression model, composed of input data, weights, a bias (or threshold), and an output. Once an input layer is determined, weights are assigned. These weights help determine the importance of any given variable, with larger ones contributing more significantly to the output compared to other inputs. All inputs are then multiplied by their respective weights and then summed. Afterward, the output is passed through an activation function, which determines the output. If that output exceeds a given threshold, it “fires” (or activates) the node, passing data to the next layer in the network. This results in the output of one node becoming in the input of the next node. This process of passing data from one layer to the next layer defines this neural network as a feedforward network. Larger weights signify that particular variables are of greater importance to the decision or outcome.

Most deep neural networks are feedforward, meaning they flow in one direction only, from input to output. However, one can also train a model through backpropagation; that is, move in the opposite direction from output to input. Backpropagation allows one to calculate and attribute the error associated with each neuron, allowing one to adjust and fit the parameters of the model(s) appropriately.

In machine learning, backpropagation is an algorithm for training feedforward neural networks. Generalizations of backpropagation exist for other artificial neural networks (ANNs), and for functions generally. These classes of algorithms are all referred to generically as “backpropagation”. In fitting a neural network, backpropagation computes the gradient of the loss function with respect to the weights of the network for a single input—output example, and does so efficiently, unlike a naive direct computation of the gradient with respect to each weight individually. This efficiency makes it feasible to use gradient methods for training multilayer networks, updating weights to minimize loss; gradient descent, or variants such as stochastic gradient descent, are commonly used. The backpropagation algorithm works by computing the gradient of the loss function with respect to each weight by the chain rule, computing the gradient one layer at a time, iterating backward from the last layer to avoid redundant calculations of intermediate terms in the chain rule; this is an example of dynamic programming. The term backpropagation strictly refers only to the algorithm for computing the gradient, not how the gradient is used; however, the term is often used loosely to refer to the entire learning algorithm, including how the gradient is used, such as by stochastic gradient descent. Backpropagation generalizes the gradient computation in the delta rule, which is the single-layer version of backpropagation, and is in turn generalized by automatic differentiation, where backpropagation is a special case of reverse accumulation (or “reverse mode”).

With respect to FIG. 3, according to exemplary embodiments, the system produces an output, which in turn produces an outcome, which in turn produces an input. In some embodiments, the output may become the input.

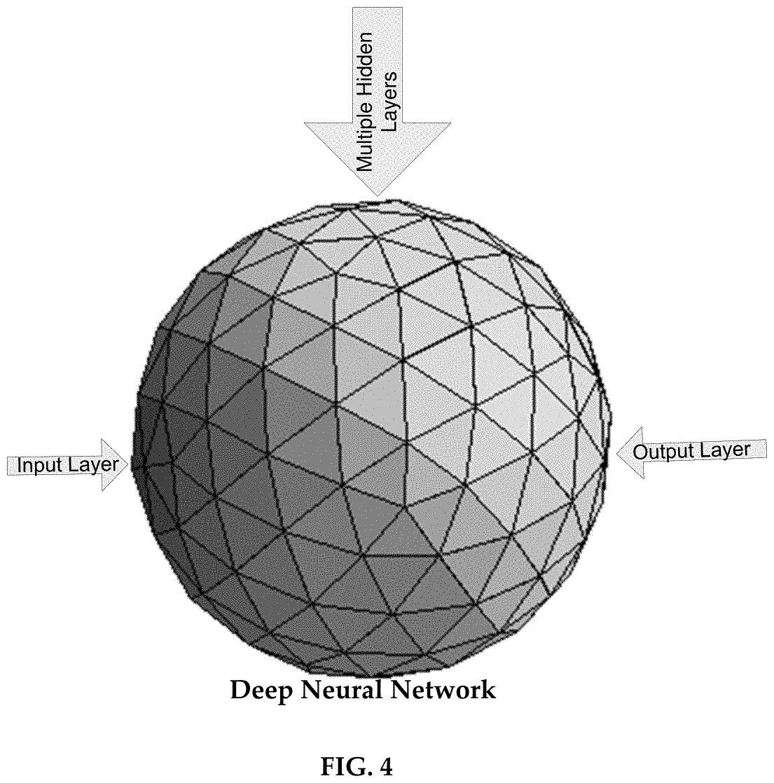

FIG. 4 shows another exemplary deep neural network in the form of a geodesic dome.

Illustrated is a dynamic multi-faceted/multi-dimensional system, having an input layer, multiple hidden layers, and an output layer. With respect to FIG. 4, according to exemplary embodiments, the system produces an output, which in turn produces an outcome, which in turn produces an input. In some embodiments, the output may become the input.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description but is not intended to be exhaustive or limited to the present disclosure in the form disclosed. Many modifications and variations will be apparent to those of ordinary skill in the art without departing from the scope and spirit of the present disclosure. Exemplary embodiments were chosen and described in order to best explain the principles of the present disclosure and its practical application, and to enable others of ordinary skill in the art to understand the present disclosure for various embodiments with various modifications as are suited to the particular use contemplated.

While various embodiments have been described, it should be understood that they have been presented by way of example only, and not limitation. The descriptions are not intended to limit the scope of the invention to the particular forms set forth herein. To the contrary, the present descriptions are intended to cover such alternatives, modifications, and equivalents as may be included within the spirit and scope of the invention as defined by the appended claims and otherwise appreciated by one of ordinary skill in the art. Thus, the breadth and scope of a preferred embodiment should not be limited by any of the above-described exemplary embodiments.

What is claimed is:

1. An intelligent secure networked health messaging system configured by at least one processor to execute instructions stored in memory, the system comprising:
a data retention system and a health analytics system, the health analytics system performing asynchronous processing with a patient's computing device and the health analytics system communicatively coupled to a deep neural network;
a web services layer providing access to the data retention and the health analytics system;
a batching service, wherein an application server layer transmits a request to the web services layer for data, the request processed by the batching service transparently to the patient;
the request processed by the batching service transparently to the patient such that the patient can continue to use a patient facing application without disruption, the patient-facing application having an audio sensor and a computer video sensor;
the application server layer including a high speed data corridor established between the application server layer and the patient's computing device that:
provides the patient-facing application that accesses the data retention and the health analytics system and the deep neural network through the web services layer;
performs processing based on patient interaction with the patient-facing application, the patient-facing application configured to execute instructions including transmitting an interactive conversational patient interface to the patient's computing device;
the deep neural network configured to:
receive a first input at an input layer;
process the first input at one or more hidden layers;
generate a first output;
transmit the first output to an output layer; and
provide the first output to the patient-facing application.

2. The intelligent secure networked health messaging system of claim 1, further comprising providing the first output to the interactive conversational patient interface.

3. The intelligent secure networked health messaging system of claim 1, further comprising the first output generating a first outcome.

4. The intelligent secure networked health messaging system of claim 3, further comprising the first outcome being transmitted to the input layer, processing the first outcome by the one or more hidden layers, generating a second output, transmitting the second output to the output layer, providing the second output to the patient-facing application and the second output generating a second outcome.

5. The intelligent secure networked health messaging system of claim 4, further comprising the second outcome being transmitted to the input layer.

6. The intelligent secure networked health messaging system of claim 1, further comprising processing a plurality of outcomes by the one or more hidden layers.

7. The intelligent secure networked health messaging system of claim 6, further comprising the processing of the plurality of outcomes for a single patient.

8. The intelligent secure networked health messaging system of claim 7, further comprising the processing determining a comorbid condition for the single patient.

9. The intelligent secure networked health messaging system of claim 6, further comprising the processing of the plurality of outcomes for a plurality of patients.

10. The intelligent secure networked health messaging system of claim 9, further comprising the processing of the plurality of outcomes for a plurality of patients having a medical condition in common.

11. The intelligent secure networked health messaging system of claim 1, further comprising the output including any of a clinically relevant care plan, a reminder, an alert, or a survey.

12. The intelligent secure networked health messaging system of claim 1, further comprising the outcome including any of a biometric parameter, a biometric parameter out of a predetermined threshold, a response to a survey, medication compliance information, an indicator of daily activity, an indicator of mood, or an indicator of stress.

13. The intelligent secure networked health messaging system of claim 1, further comprising the processing by the one or more hidden layers including using voice, speech, and computer video inputs to analyze signs of changes in health and behavioral status including but not limited to stress, anger, change in speech cadence, slurred speech or coughing.

14. The intelligent secure networked health messaging system of claim 13, further comprising the processing determining changes in health and behavioral status including but not limited to anger, substance use, lack of sleep, stress, early onset of dementia or Alzheimer's disease, an adverse reaction to a medication, a stroke, Parkinson's disease, an increased risk of falling, or a lack of balance.

15. The intelligent secure networked health messaging system of claim 1, further comprising the processing by the one or more hidden layers including using telemetry information to determine if a patient's behavior has changed in a way which could be indicative of a change in mental, emotional, or physical health and proactively inquiring before a threshold alert is triggered.

16. The intelligent secure networked health messaging system of claim 1, further comprising the patient-facing application with the interactive conversational patient interface converting response data received by the patient's computing device into an audio file using a cloud-based text-to-speech application capable of being integrated into a web browser based avatar, the avatar being displayed on a display screen within the web browser of the patient's computing device as a three-dimensional electronic image of a human caregiver for a human patient, further comprising the three-dimensional electronic image of the human caregiver providing step-by-step verbal healthcare instructions to the human patient, monitoring a response from the human patient, and providing healthcare advice to the human patient based on the response.

17. The intelligent secure networked health messaging system of claim 16, further comprising the patient-facing application configured to generate a report for a health care provider, receive instructions from the health care provider and deliver the instructions to the human patient by way of the three-dimensional electronic image of a human caregiver.

18. The intelligent secure networked health messaging system of claim 1, further comprising the processing by the one or more hidden layers including using backpropagation to compute a gradient of a loss function with respect to weights of the neural network for a single input—output.

19. The intelligent secure networked health messaging system of claim 1, further comprising the processing by the one or more hidden layers including using each individual node as its own linear regression model, composed of input data, a weight, a bias or threshold, and an output.

20. The intelligent secure networked health messaging system of claim 1, further comprising the processing by the one or more hidden layers including generating an insight on a health condition proactively before a statistically significant manifestation of a decline in the health condition.

21. The intelligent secure networked health messaging system of claim 1, further comprising the first output causing ordering a home safety inspection for a patient with noted activities of daily living limitations.

22. The intelligent secure networked health messaging system of claim 1, further comprising the first output causing ordering a functional strength examination for a patient at risk of falling.

23. The intelligent secure networked health messaging system of claim 1, further comprising the first output causing prescribing a walker or a wheelchair.
